(19) United States
(12) Patent Application Publication
(10) Pub. No.: US 2007/0100286 A1
(43) Pub. Date: May 3, 2007

(54) Needle assembly for radiating bioactive compounds through tissue

(76) Inventor: Hazem A. Eltahawy, Detroit, MI (US)

Correspondence Address: CLARK HILL, P.C., 500 WOODWARD AVENUE, SUITE 3500, DETROIT, MI, 48226, US

(21) Appl. No.: 11/264,422

(22) Filed: Nov. 1, 2005

(51) Int. Cl. A61M 5/178; A61M 25/00; A61M 5/00

(52) U.S. Cl. 604/164.12; 604/166.01; 604/264

(57) ABSTRACT

A needle assembly prepares tissue for the receipt of medicine or treatment electrodes. The needle assembly includes a body extending between a handle end and an insertion end. The body includes a hollow interior. A pointed tip is fixedly secured to the insertion end to reduce forces required to insert the needle assembly through the tissue to create a central cavity. A plurality of openings is spaced equidistantly about the body at the insertion end. The plurality of openings provides access from the hollow interior into the tissue. The needle assembly also includes a plurality of stylets extendable through the hollow body and movable axially with respect thereto. Each of the plurality of stylets exits out each of the plurality of openings to create supplemental tubular cavities radiating out from the central cavity such that the medicine forced through the hollow interior of the body will exit through the plurality of openings and into the central and supplemental cavities to more effectively distribute the medicine through the tissue.

### BACKGROUND ART

1. Field of the Invention

The invention mainly relates to needle assemblies used for injecting tissues with bioactive compounds. More specifically, the invention relates to needle assemblies capable of radiating the bioactive materials through the tissue that requires the compounds.

2. Description of the Related Art

Current techniques in transferring bioactive compounds into mammalian tissue is done through a standard needle assembly. The needle is forced through the tissue whereafter the bioactive compound is pushed through the syringe and the needle into the tissue. The technique is somewhat more complicated when the tissue is hard tissue, such as bone. In this situation, the needle assembly has to create a space in which the bone could receive the bioactive compounds (hereinafter the bioactive compounds will be referred to as medicine). To create the space in which the medicine is going to be received in the hard tissue, the needle must force itself into the hard tissue to create a type of reservoir to receive the medicine therein. With the creation of the reservoir, the medicine will fill that void and work in that specific area. Should the medicine be required in multiple locations, multiple injections will be required. This can become painful to the patient.

A type of hard tissue surgical needle is disclosed in U.S. Pat. No. 4,513,747, issued to Smith on Apr. 30, 1985. This needle defines a tip that is created by cutting edges converging together. This makes it easier to create the space in which the medicine is to be received. Once the space is created, the hard tissue surgical needle must be removed and a needle that can transfer medicine to the space is then injected into the patient. This disclosure of the hard tissue surgical needle is deficient in that each procedure requires at least two needle sticks to the patient to accomplish the transfer of medicine into the desired location. In addition, the medicine, once injected, is concentrated in a single location.

A biopsy needle is disclosed in U.S. Pat. 5,807,275 issued to Jamshidi on Sep. 15, 1998. In this reference, a single opening is disposed adjacent the tip of the needle assembly. The needle is injected into the patient, whereafter a handle is turned and a piece of tissue is retained in the tip of the needle. Once the needle is removed from the patient a biopsy may be performed on the tissue that was removed therefrom. This biopsy needle is not used for the transfer of medicine to a patient.

### SUMMARY OF THE INVENTION

A needle assembly prepares tissue for the receipt of medicine or treatment electrodes. The needle assembly includes a body extending between a handle end and an insertion end. The body includes a hollow interior. A pointed tip is fixedly secured to the insertion end to reduce forces required to insert the needle assembly through the tissue to create a central cavity. A plurality of openings is spaced about the body at the insertion end. The plurality of openings provides access from the hollow interior into the tissue. The needle assembly also includes a plurality of stylets extendable through the hollow body and movable axially with respect thereto. Each of the plurality of stylets exits out each of the plurality of openings to create supplemental tubular cavities radiating out from the central cavity such that the medicine forced through the hollow interior of the body will exit through the plurality of openings and into the central and supplemental cavities to more effectively distribute the medicine through the tissue.

### BRIEF DESCRIPTION OF THE DRAWINGS

Advantages of the invention will be readily appreciated as the same becomes better understood by reference to the following detailed description when considered in connection with the accompanying drawings, wherein:

FIG. 1 is a perspective view, partially cut away, of one embodiment of the invention being injected into hard tissue of a patient;

FIG. 2 is a perspective view, partially cut away, of a second embodiment of the invention being injected into soft tissue of a patient;

FIG. 3 is a side view, partially cut away, of an insertion end of the invention with a set of obturating stylets closing the end thereof;

FIG. 4 is a side view, partially cut away, of the insertion end of the invention with stylets extending out therefrom;

FIG. 5 is a side view, partially cut. away, of the insertion end of the invention with hollow stylets in the extended position; and

FIG. 6 is an exploded perspective view partially cut away of a third embodiment of the invention.

### DETAILED DESCRIPTION OF THE PREFERRED EMBODIMENTS

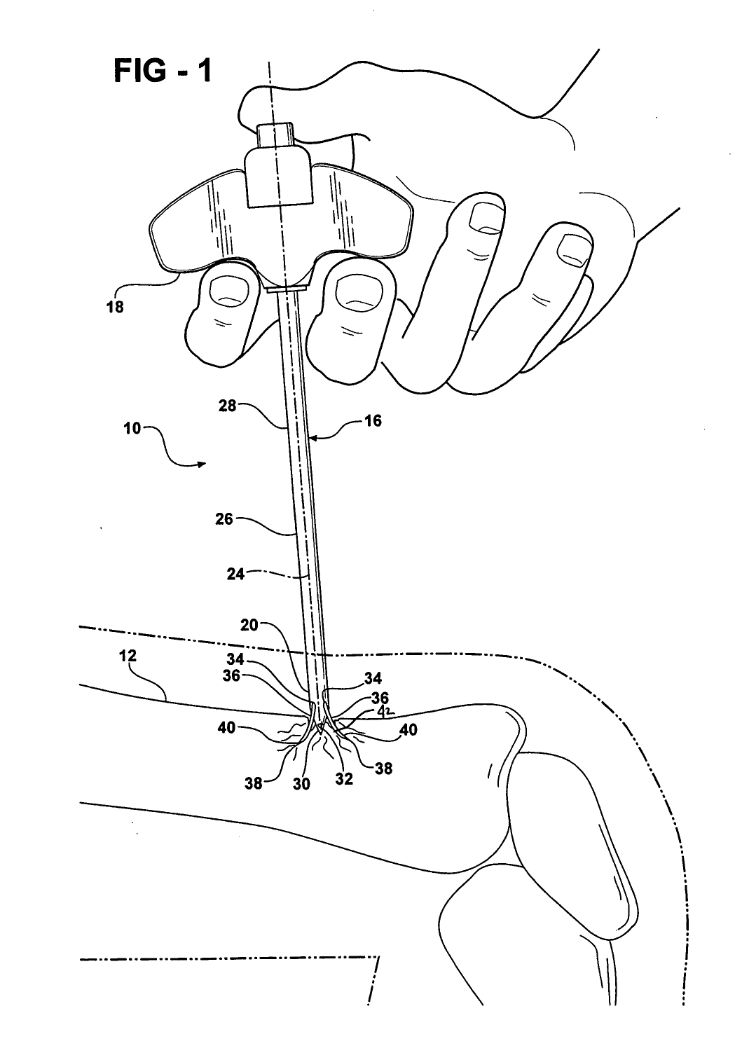

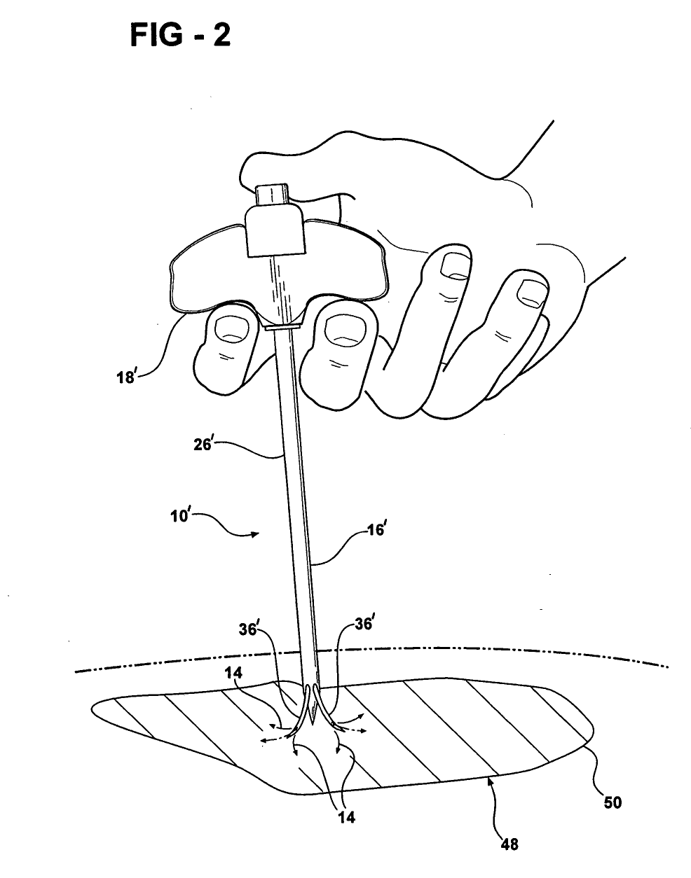

Referring to FIG. 1, a first embodiment of a needle assembly is generally indicated at 10. This needle assembly 10 is designed to prepare hard tissue 12 for the receipt of medicine (indicated by arrows 14 in FIG. 2) therein. The needle assembly 10 is designed to prepare the hard tissue, e.g. bone, by making the hard tissue 12 as accessible as possible with the least amount of injections into the patient. In addition, the needle assembly 10 is designed to distribute as much medicine 14 as uniformly as possible throughout the hard tissue 12.

The needle assembly 10 includes a body 16 that extends between a handle end 18 and an insertion end 20. The body 16 defines a hollow interior 22 that extends along the entire body 16. The body 16 defines a longitudinal axis 24 about which the hollow interior 22 is coaxial therewith. The body 16 is cylindrical and includes a cylinder wall 26 of uniform thickness extending between an exterior 28 and the hollow interior 22. For soft tissue penetration, the cylinder wall 26 may be fabricated from a translucent material such that an operator of the needle assembly 10 can see the contents of the hollow interior 22. In FIG. 1 the body 16 is fabricated from a non-translucent material, e.g., steel, so that it can effectively prepare the hard tissue 12 for the medicine 14. In this instance, a window (not shown) may extend along the cylinder side wall 26 of the body 16 so that the operator of the needle assembly 10 may still view the contents of the needle assembly 10 as they are distributed into the hard tissue 12. The diameter for the body 16 may vary depending on the use thereof. It is, however, envisaged that the diameter for human use would be 11 or 13 G.

The needle assembly 10 also includes a pointed tip 30 that is fixedly secured to the insertion end 20. The pointed tip 30 is used for penetration into the hard tissue 12. Therefore, the pointed tip 30 reduces the forces required to insert the needle assembly 10 through the hard tissue 12 to create a central cavity 32. The central cavity 32 is created inside the hard tissue 12 and will receive medicine 14 therein once the hard tissue 12 is finally prepared to receive the medicine 14.

A plurality of openings 34 extends through the cylinder side wall 26 of the body 16 at the insertion end 20. In the embodiment shown, each opening 34 is spaced equidistantly about the body 16 from each other. It should be appreciated by those skilled in the art that the openings 34 may also be concentrated in a portion of the cylinder side wall 26 to extend the stylets 36 through one aspect of a region. In addition, the locations of the openings 34 may vary along the length of the cylinder side wall 26. The plurality of openings 34 provide access from the hollow interior 22 into the hard tissue 12. In the embodiment shown, each opening 34 is oval in shape and is cut through the hollow interior 22. It should be appreciated by those skilled in the art that each opening 34 may be cut through the cylinder side wall 26 in any shape that would facilitate the function of the opening 34.

The needle assembly 10 also includes a plurality of stylets 36 that extend through the hollow body 16. The stylets 36 are removable from the body 16 when it is time to inject medicine into the hard tissue 12, should the needle assembly 10 be used as the medicine delivery mechanism. The stylets 36 move axially with respect to the body 16. Each of the plurality of stylets 36 defines a terminal end 38. The terminal end 38 includes a cutting edge 40 that aids in the passing of the stylets 36 through the openings 34 and into the hard tissue 12. The stylets 36 are fabricated from a hard material, e.g., steel, such that the stylets 36 may work through the hard tissue 12 to create channels for medicine 14 to be received therein. While it is contemplated that the stylets 36 are fabricated from a hard material, it should also be appreciated that the stylets 36 are flexible. The stylets 36 need to be flexible because they will be extending down through the hollow interior 22 of the body 16 coaxial with the longitudinal axis 24 thereof. The stylets 36 may also be made of spring retaining memory alloy with a preset bend along the shaft close to the terminal end 38 such that when forced through the hollow interior 22, they assume a straight configuration inside the hollow interior 22 and then spring back to their original bent configuration once they are forced to exit through the openings 34.

As the stylets 36 reach the openings 34, the stylets 36 will be forced to bend to exit the hollow interior 22 radially outward therefrom. When the stylets 36 move outwardly from the hollow interior 22 through the openings 34, the stylets 36 create supplemental cavities 42 that radiate out from the central cavity 32 such that medicine 14 forced through the hollow interior 22 of the body 16 will exit through the plurality of openings 34 and into the central 32 and supplemental 42 cavities to more effectively distribute the medicine 14 through the hard tissue 12. Because the stylets 36 move out of the hollow interior 22 through the openings 34, the supplemental cavities 42 will be in fluid communication with the central cavity 32, thus facilitating the transfer of medicine 14 from the central cavity 32 radially outwardly through the supplemental cavities 42 to more evenly and thoroughly distribute the medicine 14 through the hard tissue 12. While the guides 44 are shown to have the same acute angle, it should be appreciated that each of the guides 44 could have an angle different from the others to vary the direction that the stylets 36 exit the needle assembly 10.

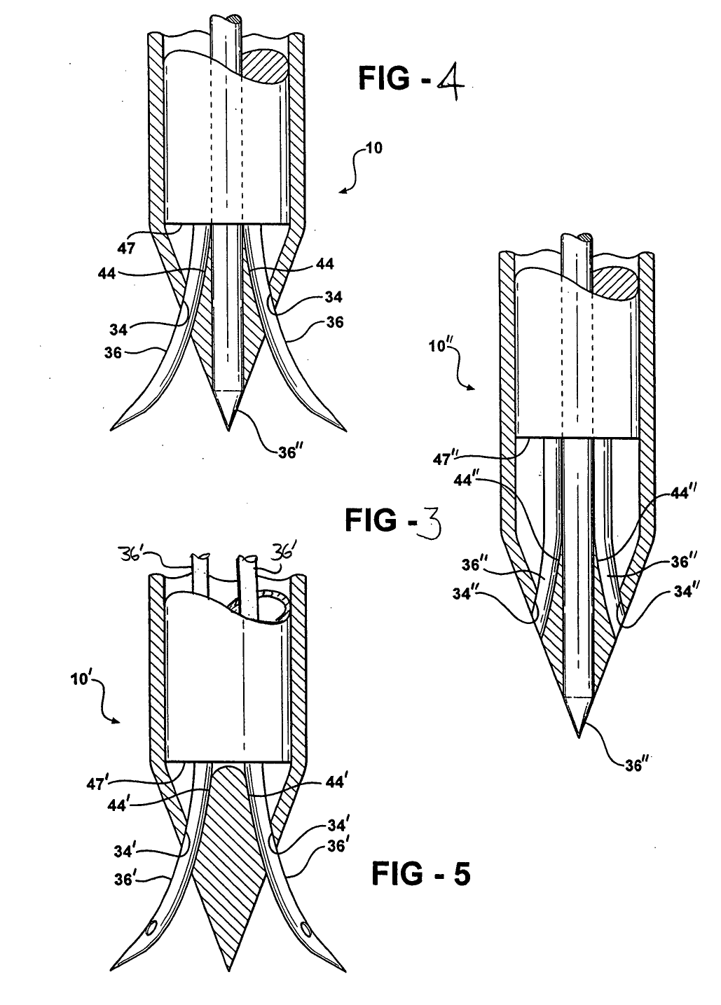

To aid in the directing of the stylets 36 out of the hollow interior, each of the plurality of openings 34 include a guide 44 (shown in FIGS. 3 and 4). The guide 44 directs each of the plurality of stylets 36 radially outwardly therefrom. Each guide 44 is a surface that extends at an acute angle with respect to the body 16. The surface 46 may extend into the hollow interior 22 beyond the cylindrical wall 26 to receive each of the plurality of stylets 36 prior to guiding the stylets 36 out of the hollow interior 22.

The needle assembly 10 is generally provided with two sets of stylets 36. The second set 36″ (shown in FIG. 4) is of the same length as the body 16 of the needle assembly and therefore do not protrude out of side openings 34. This second set 36″ acts as an obturator for the needle assembly 10 while the needle assembly 10 is plunged into hard tissue 12 to prevent its bending. A solid inner body 47 may be used with the second set of stylets 36″ secured to the end thereof. Once in place, the first set of stylets 36 replaces the second set 36″. The first set of stylets 36 is longer than the body 16 so an application of a force will have the first set of stylets 36 forced into the hard tissue 12 to create the supplemental cavities 42. More specifically, when advanced into the cylinder body 16, the first set of stylets 36 eventually will protrude out of the openings 34 and create the supplemental cavities 42. The length of the stylets 36 may be equal and therefore all protrude to the same distance outside body 16 or may be of different lengths and therefore one or more stylets may extend to a greater distance into the tissue than others depending on the anatomy of where the needle is being used.

The external ends of the stylets may be attached to a plunger 52 which constitute the movable part of the handle end 18. The fixed part of the handle 54 is attached to the handle end 18 of the body 16. Moving the plunger 52 down to be in contact with the handle 18 brings all the stylets 36 out of their side openings 34 to their maximum excursions into the tissue. The stylets 36 may be provided with graduations in centimeters or inches or another unit to mark the extent of protrusion into the tissue. Alternatively, they can be provided with a movable stopper made of a metal clip or similar arrangement that can be moved along the shaft of the stylets to stop their excursion into the tissue as the stopper comes in contact with the handle 18.

The body 16 of the needle assembly 10 may be totally hollow with the stylets 36 entering the handle end 18 and traversing the body 16 to reach the insertion end 20 where they are forced to bend freely according to the angles of the guides 44 at the side openings 34. Another embodiment, (not shown), entails that the handle end is provided with panel containing pores, at set distances to guide the entry of the stylets towards their side openings 34. In a different embodiment (also not shown), the hollow shaft of the needle is divided into hollow compartments extending between the handle 18 and the exit 20 ends. Each stylet 36 would enter through one of the compartments to reach its opening 34.

Referring to FIG. 2, a second embodiment of the needle assembly is generally indicated at 10′, wherein like prime numerals represent similar elements as those of the first embodiment, discussed above. The needle assembly 10′ is designed to be used in soft tissue, generally indicated at 48. It is contemplated that the soft tissue 48 may be a tumor in which it would be desired to have an even distribution of a medicine 14 throughout the body of the tumor defined by its outer periphery 50. In this instance, the stylets 36′ are hollow and not solid. The stylets 36′ may be fabricated from polyurethane or similar material. Each of the plurality of hollow stylets 36′ move through each of the plurality of openings 34′ to extend radially out therefrom. Once the stylets 36′ are in position, medicine is pushed through each of the plurality of stylets 36′ to radiate the medicine 14′ outwardly radially into regions in the soft tissue 48 away from the body 16′ of the needle assembly 10′. The stylets 36′ include output ports 52 disposed adjacent the cutting edge 40′ of the terminal end 38′. The output ports 52 allow the medicine 14′ to move out from the stylets 36′ into the supplemental cavities 42′ created by the stylets 36′.

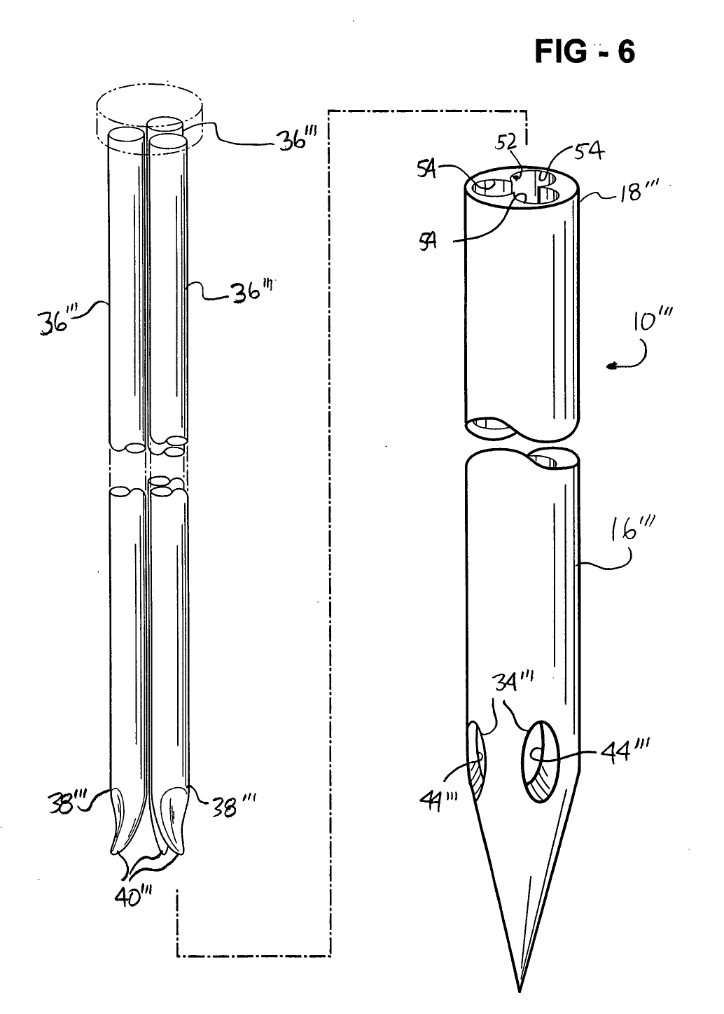

Referring to FIG. 6, a third embodiment of the invention is generally indicated at 10′″. The needle assembly 10′″ has a body 16′″ with a non-cylindrical inner wall, generally shown at 52. The inner wall defines a plurality of lobes 54. Each of the plurality of lobes 54 receives one of the stylets 36′″ therein. It should be appreciated by those skilled in the art that the number of lobes 54 and the number of stylets 36′″ may vary depending on the design of the needle assembly 10′″.

The lobes 54 may extend down the entire length of the body 16′″. Alternatively, the plurality of lobes 54 may only exist at the handle end 18′″thereof. In fact, the plurality of lobes 54 may be created in a cap-like structure (not shown) that may be inserted over the top of the handle end 18′″ of the body 16′″. Each of the lobes 54 aides in the maintenance of the stylet 36′″ in a particular position with respect to the body 16′″ so that it may be received by a lumen 34′″ in a manner which will not require the stylets 36′″ to be rotated once inside the body 16′″ to ensure that each of the stylets 36′″ are capable of being pushed through a lumen 34′″.

The invention has been described in an illustrative manner. It is to be understood that the terminology, which has been used, is intended to be in the nature of words of description rather than of limitation.

Many modifications and variations of the invention are possible in light of the above teachings. Therefore, within the scope of the appended claims, the invention may be practiced other than as specifically described.

1. A needle assembly for preparing hard tissue for the receipt of medicine, said needle assembly comprising:
a body extending between a handle end and an insertion end, said body having a hollow interior;
a pointed tip fixedly secured to said insertion end to reduce forces required to insert said needle assembly through the hard tissue to create a central cavity;
a plurality of openings spaced about said body at said insertion end providing access from said hollow interior out into the hard tissue; and
a plurality of stylets extendable through said hollow interior and movable axially with respect thereto, wherein each of said plurality of stylets is solid and moves through each of said plurality of openings to create supplemental cavities radiating out from the central cavity such that the medicine forced through said hollow interior of said body will exit through said plurality of openings and into the central and supplemental cavities to more effectively distribute the medicine through the hard tissue.

2. A needle assembly as set forth in claim 1 wherein each of said plurality of openings includes a guide for directing each of said plurality of stylets radially outward therefrom.

3. A needle assembly as set forth in claim 2 wherein each of said guides includes a surface extending inwardly into said hollow interior at an acute angle with respect to said body.

4. (canceled)

5. A needle assembly as set forth in claim 3 wherein each of said plurality stylets is flexible such that each of said plurality of stylets is redirected by each of said guides as said plurality of stylets is moved down through said hollow body.

6. A needle assembly as set forth in claim 5 wherein said plurality of stylets is fabricated from steel.

7. (canceled)

8. (canceled)

9. (canceled)

10. (canceled)

11. (canceled)

12. (canceled)

13. (canceled)

14. (canceled)

15. (canceled)

16. A method for transmitting bioactive material to hard tissue of a body using a needle assembly having a hollow interior, a pointed tip, a plurality of holes disposed adjacent the pointed tip, and a plurality of stylets, the method comprising the steps of:
puncturing the body using the pointed tip of the needle assembly;
pushing the needle assembly through the body and into the hard tissue thereof to create a central cavity;
forcing the plurality of stylets out of the needle assembly through a plurality of holes into the hard tissue to create supplemental cavities in the hard tissue;
retracting the plurality of stylets from the hard tissue; and
injecting the bioactive material into the central cavity and the supplemental cavities after the plurality of stylets have been retracted into the needle assembly.
